(19) United States
(12) Patent Application Publication
(10) Pub. No.: US 2025/0302465 A1
(43) Pub. Date: Oct. 2, 2025

(54) DUAL GUIDE INSERTER FOR DEPLOYING ANCHORS IN KNOTLESS REPAIR

(71) Applicant: Biomet Manufacturing, LLC, Warsaw, IN (US)

(72) Inventors: Ryan A. Kaiser, Leesburg, IN (US); Nyree Masoian, San Ramon, CA (US)

(21) Appl. No.: 19/087,932

(22) Filed: Mar. 24, 2025

Related U.S. Application Data
(60) Provisional application No. 63/570,461, filed on Mar. 27, 2024.

(51) Int. Cl. A61B 17/04; A61B 17/17

(52) U.S. Cl. CPC A61B 17/0401; A61B 17/17; A61B 2017/0409; A61B 2017/0464

(57) ABSTRACT

A tissue repair device is discussed and illustrated herein. The tissue repair device can include a handle, a shaft assembly coupled to the handle, a distal tip portion and an actuator. The shaft assembly can have a first lumen and a second lumen. The first lumen can be offset from and extends adjacent but separate from the second lumen. The first lumen can be configured to selectively receive a drill. The distal tip portion can be configured to selectively carry a deformable anchor. The distal tip portion can have a guide. The actuator can be moveably coupled to the handle and the shaft assembly. A pusher of the actuator can be moveably received within the second lumen and can engage the deformable anchor and advance the deformable anchor distally from the distal tip portion as directed by the guide.

### CLAIM OF PRIORITY

This application claims the benefit of U.S. Provisional Patent Application Ser. No. 63/570,461, filed on Mar. 27, 2024, the benefit of priority of which is claimed hereby, and which is incorporated by reference herein in its entirety.

### FIELD OF THE DISCLOSURE

The present disclosure relates to devices used for bone or tissue repair surgery including a device for guiding drilling and for inserting a suture anchor for such repair.

### BACKGROUND OF THE DISCLOSURE

In the human body, tissue can require repair. Such tissue includes bone, muscles, tendons, ligaments and cartilage. Forceful twisting, trauma or rotation of the knee, shoulder (or other joint) can tear or otherwise damage tissue. A surgical repair of the tissue may be required. Such repair can include suturing and anchoring to bone. Various assemblies have been developed for facilitating suturing and are effective for their intended purposes. Nevertheless, improvements to tissue repair devices for facilitating suturing are still desirable.

### SUMMARY

Tissue repair devices (commonly called inserters) are used for suture anchor deployment into tissue (including bone and/or soft tissue). Although inserters have been developed are generally effective, some tissue repairs such a labral repair are complex and require the deployment of multiple anchors to anchor sutures and repair tissue in multiple locations.

The present inventors have recognized that surgical complexity and procedure time can be reduced with the present tissue repair devices, which can be configured to act as both a drill guide and deployment device for the suture anchor. Many inserters presently utilized are not configured for such dual use. As such, one tool must be used for guiding the drill and then another tool is used as the inserter for deployment of the suture anchor. This process can be tedious as the inserter and drill guide must be swapped back and forth for deployment and drilling at the multiple locations required for the repair.

The present inventors have further recognized that surgical complexity and procedure time can further be reduced as the present tissue repair devices are configured to drill tissue and deploy the suture anchor at single location without having to withdrawn from the anatomy between drilling and deployment. Thus, a surgeon need not have to perform drilling and then withdraw the tissue repair device to load a suture onto the inserter and then place the inserter back in the desired anatomical location. Rather, with the present tissue repair devices these can remain in situ as the suture anchor can be preloaded onto the tissue repair device as present repair devices are configured with a drill guide lumen that is offset from the suture anchor.

### BRIEF DESCRIPTION OF THE DRAWINGS

FIG. 1 illustrates a labral repair being performed on a shoulder joint of a patient in accordance with one example of the present application.

FIG. 2 is a view, partially in section, of an exemplary labral repair surgical technique using a tissue repair device as a guide for an exemplary drill according to one example of the present application.

FIG. 3 is a schematic view of the tissue repair device with an exemplary suture and deformable anchor coupled thereto according to one example of the present application.

FIG. 4 is a perspective view of an exemplary deformable anchor slidably coupled to the suture of FIG. 3 according to one example of the present application.

FIG. 5 shows the tissue repair device with the deformable anchor coupled thereto, wherein the tissue repair device is configured as a drill guide and to deploy the deformable anchor in accordance with one example of the present application.

FIG. 5A is a cross-sectional view of the tissue repair device of FIG. 5.

FIGS. 5B and 5C are enlarged views of a distal tip portion and a guide of the tissue repair device of FIG. 5.

FIG. 5D is an enlarged cross-sectional view of the distal tip portion of the tissue repair device of FIG. 5A.

FIG. 6 is a perspective view of the tissue repair device of FIGS. 5-5D from a proximal side thereof.

FIG. 7 is a perspective view of the tissue repair device of FIGS. 5-6 with a pusher engaging the deformable anchor and the pusher advancing the deformable anchor distally from the distal tip portion as directed by the guide which is engaged by the pusher when the pusher is sufficiently advanced.

FIGS. 8A and 8B are perspective views showing a first shaft, a second shaft and a distal tip portion of another tissue repair device according to one example of the present application.

FIG. 9 is a cross-sectional view of the first shaft, the second shaft and the distal tip portion of the tissue repair device of FIGS. 8A and 8B.

FIG. 10 is a cross-sectional view of the distal tip portion of the tissue repair device of FIGS. 8A and 8B.

FIG. 11 shows a flow diagram of a method of deploying a deformable anchor from a tissue repair device according to one example of the present application.

Corresponding reference characters indicate corresponding parts throughout the several views. Inserters in the drawings are not necessarily drawn to scale. The configurations shown in the drawings are merely examples, and should not be construed as limiting the scope of the invention in any manner.

### DETAILED DESCRIPTION

To repair tissue (including both soft tissue and bone) in the human body a surgeon can deploy two or more suture anchors into the tissue. Suture anchors and suture can be used to reattach muscle to bone, for example. Although the following description is related generally to instruments, sutures and suture constructs using deformable anchors in connection with a shoulder labral repair technique, it will be understood that the devices and methods discussed herein can also be applicable to other appropriate surgical procedures, such as, for example, a labral tear in a hip joint. Therefore, it will be understood that the following discussions are not intended to limit the scope of the present teachings or claims herein.

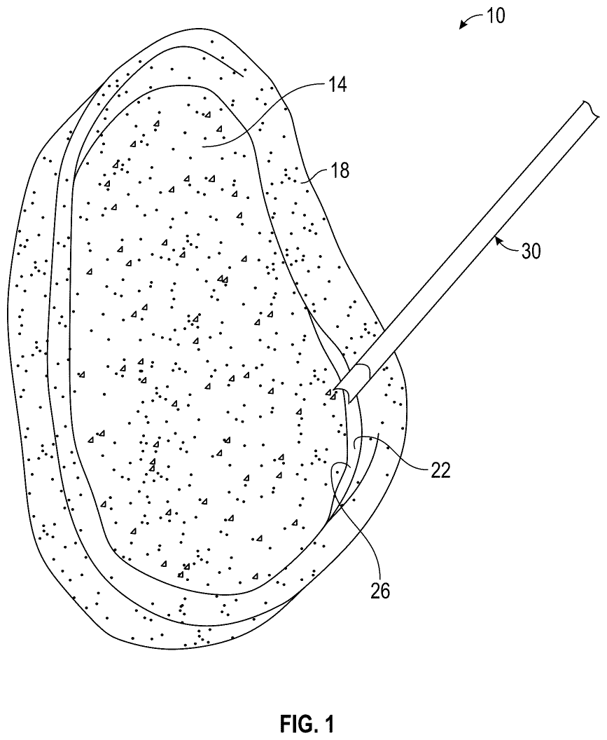

FIG. 1 illustrates a portion of an exemplary shoulder joint 10 is shown with a glenoid 14 and a labrum 18 attached thereto. The illustrated shoulder joint 10 includes an exemplary labral tear 22, such as a Bankart labral tear, where a portion of the labrum 18 has been separated from a surface 26 of the glenoid 14.

FIG. 1 shows an exemplary method of repairing labral tear 22 along with exemplary instruments and suture constructs will now be discussed. Once the labral tear 22 has been identified, the portion of the labrum 18 associated with labral tear 22 can be held away or positioned offset from the glenoid surface 26 to expose the underlying bone surface where the labrum 18 will be attached. The bone surface can be prepared as bleeding bone surface with any appropriate tool, such as a rasp (not shown). The labrum 18 can be held away from surface 26 with a suitable instrument. A tissue repair device 30 with a distal portion can be inserted into the anatomy at or adjacent the labral tear 22 as illustrated.

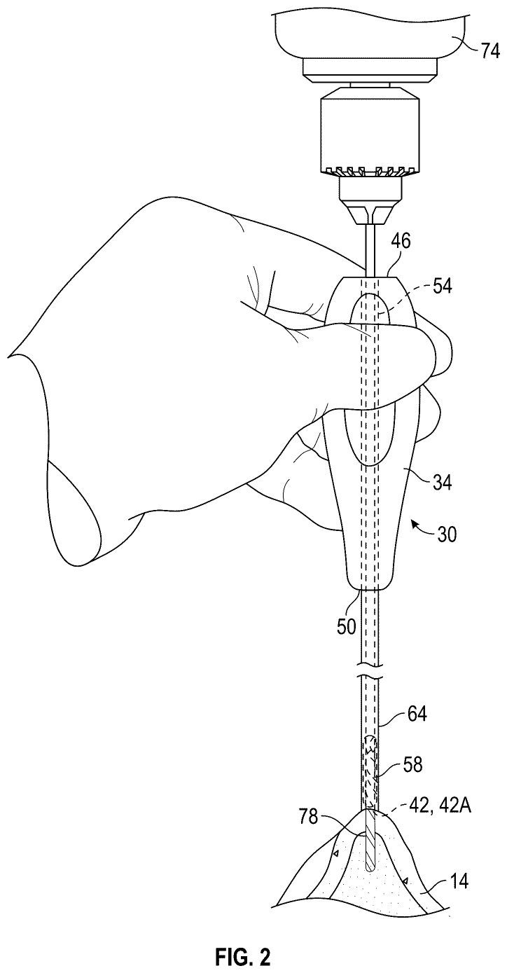

As shown in FIG. 2, the tissue repair device 30 can include a proximal handle 34 and an elongated cannulated shaft assembly 38 extending from handle 34 to a distal tip portion 42. The handle 34 can include a proximal end 46, a second opposite distal end 50 and a first lumen 54 aligned with an passing along a first shaft 64 of the shaft assembly 38. The first lumen 54 or another feature can be configured to guide a drill bit 58 as further discussed below.

As shown in FIG. 2, distal tip portion 42 of the tissue repair device 30 can be positioned as desired. Distal tip portion 42 will be constructed as further discussed and illustrated herein. For example, the distal tip portion 42 can include a guide 42A (e.g., a recess, bend, prong(s), curvature ramp and/or U-shaped configuration) to aid in deflecting the drill bit 58 for reaching the labral tear 22 and/or to access the glenoid 14 or other bone. Once the tissue repair device 30 is positioned as desired, the drill bit 58 can be coupled to a drill or driver 74. The drill bit 58 can be inserted into the first lumen 54 and can be directed by the guide 42A (e.g., bent or deflected thereby) to drill a bore 78 in the glenoid bone 14 for receipt of a deformable anchor, as will be discussed in greater detail below. Drill bit 58 can be appropriately sized relative to a length of the tissue repair device 30 so that a specific length bore can be drilled into the glenoid 14. In particular, the drill can be advanced relative to the tissue repair device 30 with aid and direction of the guide 42A of the tissue repair device 30. According to one example, the drill bit 58 can be constructed of a material the is deflectable, bendable and/or curvable such as nitinol or other material(s) such that the drill bit 58 can be deflected by the guide 42A of the tissue repair device 30 as further described herein. As illustrated, the guide 42A can be selectively engaged by the drill bit 58 when the drill bit 58 is sufficiently advanced, whereby the guide 42A causes a bending of the drill (the drill bit 58) along a lateral path that intersects with a path of a pusher (path and pusher shown in FIG. 7) when the pusher is sufficiently advanced to selectively engage the guide 42A. Additionally, various drill bits having different lengths can be provided for selection by a surgeon to drill an appropriate length bore in the glenoid of a patient. Upon drilling bore 78, the drill 74 and drill bit 58 can be removed from the tissue repair device 30 while maintaining the tissue repair device 30 in the same position over the bore 78 for deploying a deformable anchor as discussed subsequently.

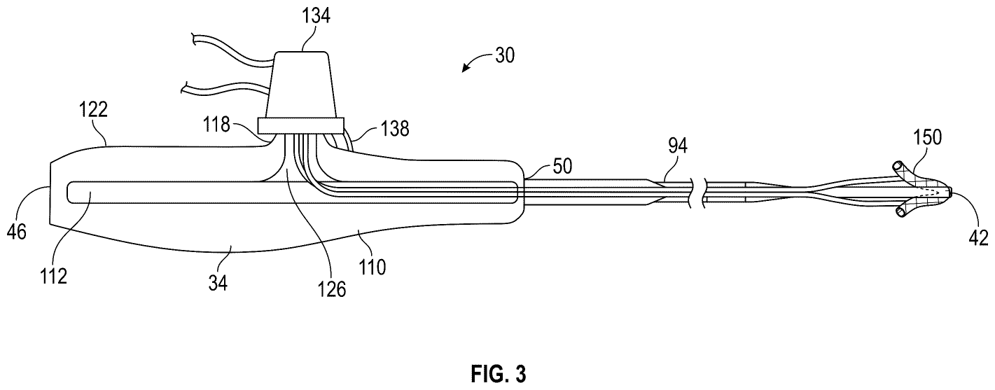

FIG. 3 shows a schematic view of the tissue repair device 30, which is configured as an inserter instrument as well as the drill guide discussed in reference to FIG. 2. The tissue repair device 30 can be configured to facilitate implanting various anchors including the deformable anchor discussed subsequently and suture constructs into a prepared bore in the anatomy, such as the bore 78 (FIG. 2).

The tissue repair device can include the handle 34, the shaft assembly 38 including a second shaft 94 extending therefrom along a longitudinal axis of the instrument. The handle 34 can include the first end 46 and the second end 50 as discussed previously. The handle 38 can include a longitudinal recess or channel 112 extending along at least a portion of a longitudinal length of handle 34. A protrusion 118 can extend vertically from an outer surface 122 of handle 90 or perpendicular to the longitudinal axis and can include a slot 126 or opening on a side 110 of handle 90 having longitudinal channel 112, which the slot 126 communicates with channel 112. A cap 134 or securing member can be coupled to handle 38 via a strap 138 and can be configured to removably engage a protrusion 118 of the handle 38 to secure a suture construct to the handle 38.

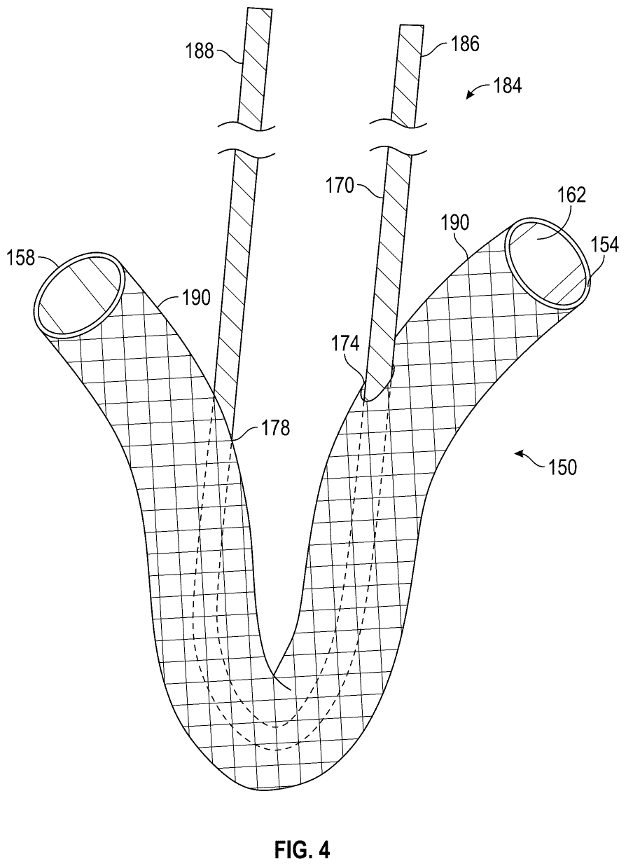

The second shaft 94, which is part of the shaft assembly 38, can extend from the second end 50 and can couple to or can form part of the distal tip portion 42 as further described and illustrated in detail subsequently. The distal tip portion 42 can be sized and shaped to removably capture (e.g., receive) a deformable anchor 150, as shown in FIG. 3. The deformable anchor 150 can be an elongate member having a sleeve or tubular configuration with a first end 154 and a second end 158 and an internal passage 162 extending therebetween, as shown in FIG. 4. The deformable anchor 150 can be made of resorbable or non-resorbable materials, including braided suture, sponges and sponge-like materials in solid form, perforated materials, woven/braided from biocompatible materials or fibers, such as, for example, polymer, polyester, polyethylene, cotton, silk, or other natural or synthetic materials.

As shown in FIG. 4, the deformable anchor 150 can have any properties that allow the deformable anchor 150 to change shape (e.g., flex, collapse, etc.). In this regard, the deformable anchor 150 can be, for example, compliant, flexible, foldable, squashable, squeezable, deformable, limp, flaccid, elastic, low-modulus, soft, spongy or perforated, or have any other characteristic property that allows it to change shape. In some aspects, the deformable anchor 150 can be coated with biological or biocompatible coatings, and also can be soaked in platelets and other biologics, which can be easily absorbed by the deformable anchor 150. In one exemplary configuration, the deformable anchor 150 can be formed from a strand of No. 5 braided polyester suture. The deformable anchor 150 can be made of resorbable or non-resorbable materials, including braided suture, sponges and sponge-like materials in solid form, perforated materials, woven/braided from biocompatible materials or fibers, such as, for example, polymer, polyester, polyethylene, cotton, silk, or other natural or synthetic materials, including sponges and sponge-like materials. In other words, multiple fibers can be braided together to form a hollow braided suture having a longitudinal passage. The deformable anchor 150 can also be referred to herein as a soft suture anchor(s). The deformable anchor 150 is referred to as “soft” herein as they are formed of material(s) that are flexible and/or deformable, such as a suture sleeve or other suture material. These soft suture anchors can be constructed as tubes that are collapsible once tension is applied to the suture. Use of soft suture anchors made of deformable material can be preferable to using a hard-plastic anchor, made from a relatively hard material, such as polyether ether ketone (PEEK). For example, in cases where the suture pulls through the tissue, the suture anchor can be dislodged within the joint or surgical space. In such instances, chances of damage is reduced with the soft suture anchor as compared with the hard suture anchor. Soft anchors are known and include the JuggerKnot® Soft Anchor manufactured and sold by Zimmer Biomet of Warsaw, IN.

As shown in FIG. 4, a suture 170 can be passed through a first opening 174 in a wall of the deformable anchor 150, guided into and along the passage 162, and passed out of the passage 162 through a second opening 178 in a wall of the deformable anchor 150 to form a suture construct 184 having free ends 186 and 188. The openings 174, 178 can be positioned intermediately between the first and second ends 154, 158 of the deformable anchor 150 at a distance of, for example, one-quarter length from ends 154, 158. It will be appreciated that the openings 174, 178 can be apertures or voids in the woven fabric of the deformable anchor 150, such that the openings 174, 178 do not disrupt or break the weave of the deformable anchor 150 when made of braided or woven material. Further, portions of the deformable anchor 150 between the first and second ends 154, 158 and the corresponding first and second openings 174, 178, can define anchoring leg or tail portions 190 that can provide additional resistance for securing the deformable anchor 150 relative to the bone, as will be discussed in greater detail herein. In one exemplary configuration, suture 170 can pass only through openings 174, 178 and a portion of the passage 162 extending therebetween to form a loop that does not extend through tail portions 190. Other configurations for the deformable anchor 150 are contemplated and the configuration of FIG. 4 is provided merely for exemplary purposes.

Tissue repair devices for deploying soft anchors connected with the suture loop, such as those of application Ser. No. 15/482,106 (now U.S. Pat. No. 10,499,902), application Ser. No. 15/654,386 (now U.S. Pat. No. 10,695,045), application Ser. No. 16/251,342 (now U.S. Pat. No. 11,116,495), application Ser. No. 14/055,172 (now U.S. Pat. No. 9,724,090) and application Ser. No. 18/133,848 are known. The contents of these applications are incorporated herein by reference in their entirety. At least application Ser. No. 18/133,848 can utilize the JuggerKnot® Soft Anchor preloaded on the tissue repair device. The present application provides improvements upon the tissue repair devices of these applications for deploying suture anchors and guiding drilling.

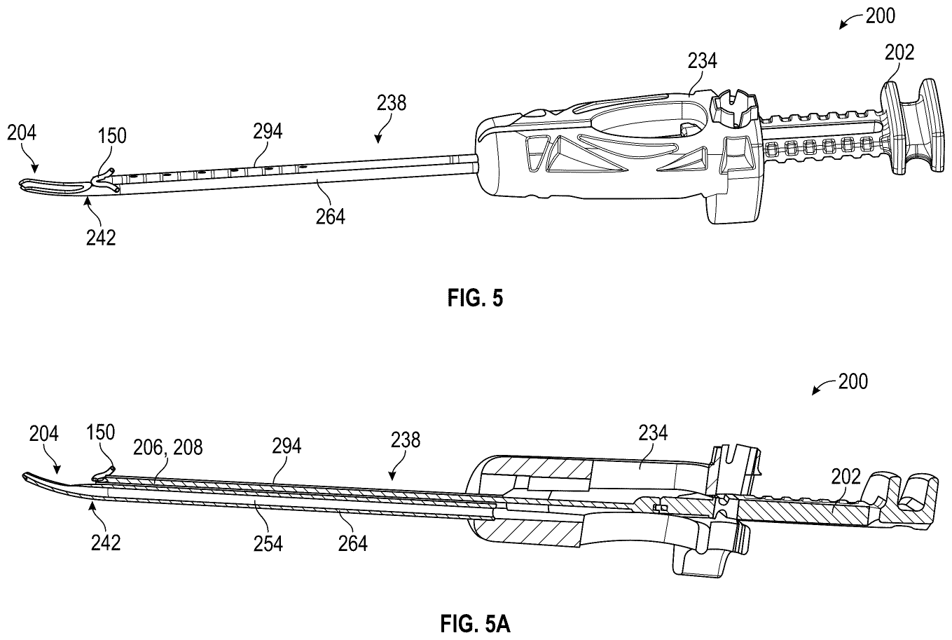

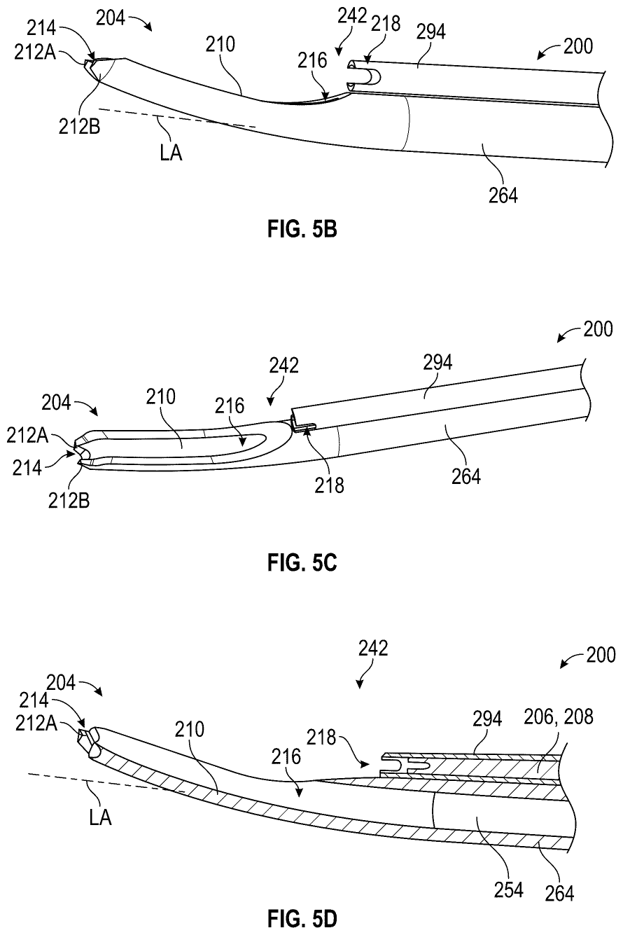

FIGS. 5 and 5A show an example of a tissue repair device 200 constructed in a manner similar to the tissue repair device 30 discussed previously. The tissue repair device 200 can include a handle 234, a shaft assembly 238, a distal tip portion 242 and an actuator 202. The shaft assembly 238 can include a first shaft 264 and a second shaft 294. The distal tip portion 242 can include a guide 204. The tissue repair device 200 of FIGS. 5 and 5A and FIG. 7 shows the deformable anchor 150 preloaded thereon. However, the sutures attached to the deformable anchor 150 are not specifically illustrated with the understanding the sutures would be connected in the manner described in reference to FIGS. 3 and 4, for example.

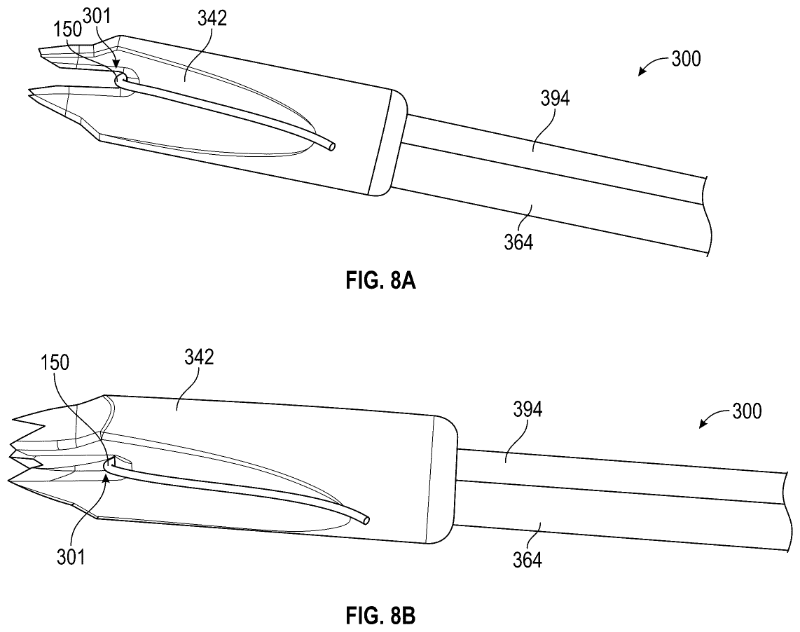

The handle 234 can include various components and can be constructed in the manner described previously. Some components such as the cap 134 (FIG. 3) are removed in FIG. 5. The handle 234 can be configured to receive the actuator 202 at least partially therein. The actuator 202 can be moveably coupled to the handle 234 and the shaft assembly 238. The shaft assembly 238 can be coupled to the handle 234 at a distal end thereof. The shaft assembly 238 can have a elongate length extending from the handle 234 to the distal tip portion 242. The first shaft 264 can be offset from and can extend adjacent but separate from the second shaft 294. The shaft assembly 238 can form the distal tip portion 242 according to some examples (such as the one shown in FIGS. 5 and 5A). However, according to other examples such as those illustrated in FIGS. 8A-10, the distal tip portion 242 can be a dedicated component that is separate from the shaft assembly.

The first shaft 264 can differ in size and/or length than the second shaft 294. As an example, the first shaft 264 can be larger in diameter, relatively longer and can be configured to receive a portion of the drill (FIG. 2) such as the drill bit as previously discussed and illustrated. The second shaft 294 can be configured to receive a portion of the actuator 202 therein as further described and shown subsequently.

As shown in FIG. 5A, the first shaft 264 can be cannulated and can have a first lumen 254. The second shaft 294 can be cannulated and can have a second lumen 206. The first lumen 254 can be offset from and can extend adjacent but separate from the second lumen 206. The first lumen 254 can be configured to selectively receive the drill bit (FIG. 2) as previously discussed. Additionally, as shown in FIG. 5A, the actuator 202 can include a pusher 208. The pusher 208 can be a wire, shaft or other component that is moveably received within the second lumen 206. The pusher 208 is configured to engage the deformable anchor 150 such as via prongs, groove(s), arms, teeth or a combination of features as known in the art.

As shown in FIGS. 5 and 5A, the distal tip portion 242 can be located at or adjacent the distal end of the tissue repair device 200 (e.g., formed by distal end and or distal end portions of the first shaft 264 and/or the second shaft 294). The distal tip portion 242 can be configured to selectively carry the deformable anchor 150. Thus, the deformable anchor 150 can be loaded onto the tissue repair device 200 even prior to drilling operations described in reference to FIG. 2 according to some examples. Thus, the tissue repair device 200 can be configured for both guiding drilling and for deploying the deformable anchor 150 into a cavity in tissue created by the drilling. The deformable anchor 150 can be held by the distal tip portion 242 such that the deformable anchor 150 is offset from the first shaft 264 but can be generally aligned with a distal end of the second shaft 294. The guide 204 can be distal of the deformable anchor 150 and will be discussed in further detail with reference to FIGS. 5B-5D.

FIGS. 5B-5D show various enlarged views of the tissue repair device 200, in particular, parts of the first shaft 264, the second shaft 294 and the distal tip portion 242. For simplicity in FIGS. 5B-5D, the deformable anchor 150 (FIGS. 5 and 5A) is not illustrated. The distal tip portion 242 can include the guide 204. The guide 204 can include a ramp 210, a first finger 212A, a second finger 212B, a first opening 214 and a second opening 216.

The guide 204 can be positioned distal of a capture feature 218 of the second shaft 294. The capture feature 218 is configured to retain and hold the deformable anchor 150 (FIGS. 5 and 5A) therein. The capture feature 218 can be prongs separated by a slot, for example. The ramp 210 can begin adjacent to and distal of the capture feature 218. The ramp 210 can be formed by a distal part of the first shaft 264, for example. However, other examples contemplate the ramp formed by another dedicated component. The ramp 210 can be arcuately curved along a longitudinal length (e.g., proximal-distal) and can additionally be arcuately curved or otherwise shaped in a lateral direction. However, other shapes for the ramp 210 are also contemplated (e.g., curved in three-dimensions, substantially straight, etc.). The ramp 210 can include an elongate opening along the length thereof allowing the physician to visualize the position of the drill and/or pusher during operation. According to one example, the ramp 210 can be formed by a portion of first shaft 264 that is shaped in a similar manner to at least part of the first lumen 254 (FIG. 5D). Thus, at least a portion of the shape of the first lumen 254 (FIG. 5D) can be replicated with the shape of the ramp 210. The ramp 210 can communicate with the first lumen 254 (FIG. 5D) via the first opening 214. The ramp 210 can extend from the first opening 214 longitudinally (e.g., proximal-distal and curved relative to axis LA of the first shaft 264 as shown in FIGS. 5B and 5D) to the second opening 216. The second opening 216 can be formed between the first finger 212A and the second finger 212B. The first finger 212A and the second finger 212B can be configured (e.g., having sharps) to provide for stability of the distal tip of the tissue repair device 200 against tissue during deployment of the deformable anchor 150 (see FIG. 7) and drilling (see e.g., FIG. 2).

The curvature or other shape of the ramp 210 of the guide 204 can deflect (e.g., curve or bend) the drill in a desired manner during drilling operation described previously in regard to FIG. 2). Additionally, the guide 204 (via the ramp 210, the first finger 212A, the second finger 212B and the second opening 216) can be configured to be engaged by the pusher 208 (FIG. 5D) during operation when the pusher 208 (FIG. 5D) is advanced sufficiently to engage with the guide 204 as illustrated for example in FIG. 7. Thus, as shown in FIG. 7, the pusher 208 (FIGS. 5A, 5D and 7) is configured to engage the deformable anchor 150 (FIGS. 5A and 7) and advance the deformable anchor 150 (FIGS. 5A and 7) distally from the distal tip portion 242 as directed by the guide 204 which is engaged by the pusher 208 (FIGS. 5A, 5D and 7) when the pusher 208 (FIGS. 5A, 5D and 7) is sufficiently advanced as shown for example in FIG. 7.

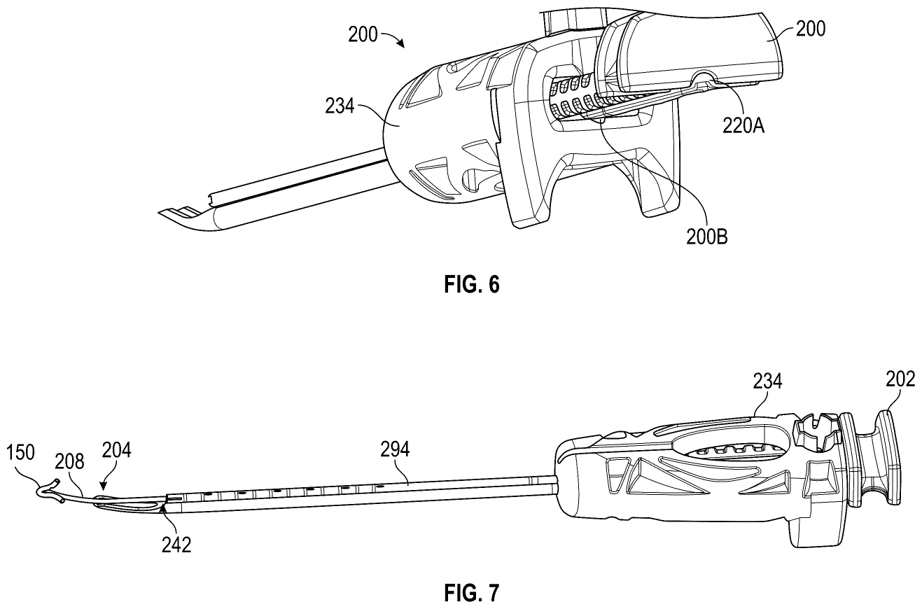

FIG. 6 shows the tissue repair device 200 from a proximal side and additionally shows the handle 234 and the actuator 202 can include passages 220A and 220B, respectively. The passages 220A and 220B can be configured to selectively receive and guide the drill (see FIG. 2) into the first lumen 254 (FIGS. 5A and 5D). Thus, the handle 234 and/or the actuator 202 is configured to selectively receive and guide the drill into the first lumen 254 (FIGS. 5A and 5D).

FIG. 7 shows the tissue repair device 200 with the actuator 202 moved distally relative to the handle 234 from the position previously illustrated in FIGS. 5-6. The advancing of the actuator 202 distally as shown in FIG. 7 advances the pusher 208 from the second shaft 294 and engages the pusher 208 with the deformable anchor 150. It should be noted that according to some examples, the deformable anchor 150 can be preloaded (e.g., engaged with) the pusher 208 prior to the deployment movement shown in FIG. 7. Thus, for example, the pusher 208 can already engage the deformable anchor 150 when the actuator 202 is positioned in the arrangement of FIGS. 5-6. As shown in FIG. 7, the pusher 208 engages the deformable anchor 150 and advances the deformable anchor 150 distally from the distal tip portion 242 as directed by the guide 204 which is engaged by the pusher 208 when the pusher 208 is sufficiently advanced for engagement to occur. Recall additionally that in FIG. 2, the guide 204 is selectively engaged by the drill when the drill is sufficiently advanced, whereby the guide 204 causes a bending of the drill along a lateral path that intersects with a path of the pusher 208 (path shown in FIG. 7) when the pusher 208 is sufficiently advanced to selectively engage the guide 204.

FIGS. 8A and 8B are perspective views showing a first shaft 364, a second shaft 394 and a distal tip portion 342 of another tissue repair device 300. The distal tip portion 342 is illustrated capturing and retaining the deformable anchor 150 with a slot 301. However, for simplicity the sutures connected to the deformable anchor 150 are not illustrated with the understanding that such sutures would be connected in the manner described in reference to FIGS. 3 and 4. In the example of FIGS. 8A and 8B, the distal tip portion 342 is a separate component from the first shaft 364 and the second shaft 394. The distal tip portion 342 comprises a distinct assembly that couples the first shaft 364 with the second shaft 394. The distal tip portion 342 can be constructed so as to be enlarged relative to the first shaft 364 and the second shaft 394. This allows the distal tip portion 342 to be more robust than the distal tip portions previously illustrated and described.

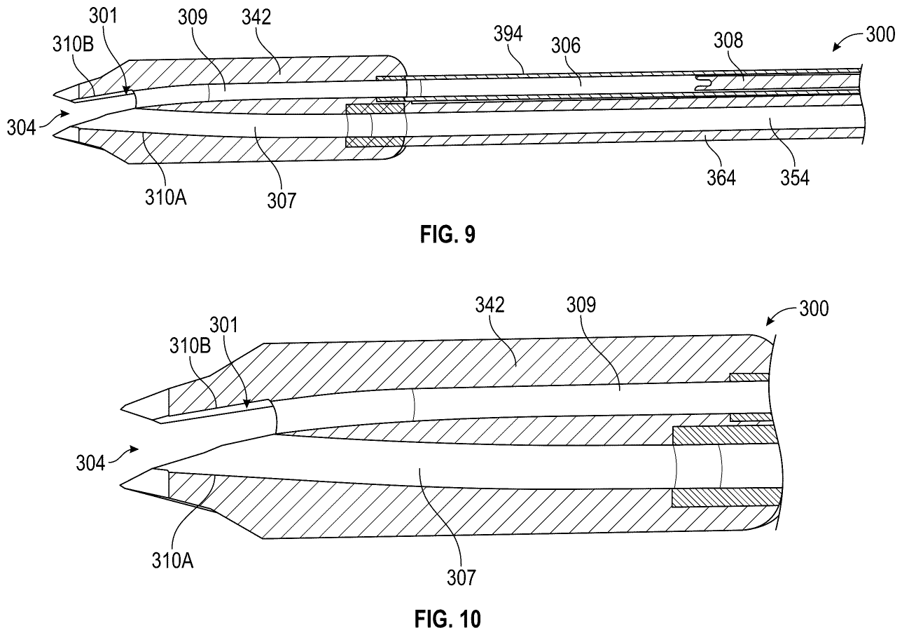

FIG. 9 is a cross-sectional view of the first shaft 364, the second shaft 394 and the distal tip portion 342 of the tissue repair device 300. FIG. 10 is an enlarged cross-sectional view of the distal tip portion 342. The deformable anchor 150 (FIGS. 8A and 8B) is not shown in FIGS. 9 and 10 for clarity.

As shown in FIG. 9, the first shaft 364 includes a first lumen 354. The second shaft 394 includes a second lumen 306. The distal tip portion 342 can include a guide 304. The guide 304 can include a first ramp 310A and a second ramp 310B. The distal tip portion 342 can additionally include a first passage 307 and a second passage 309. FIG. 9 additionally illustrates a pusher 308 moveably positioned within the second lumen 306 of the second shaft 394.

As shown in FIG. 9, the first passage 307 can communicate with the first lumen 354. The second passage 309 can communicate with the second lumen 306. The first passage 307 can at a distal portion thereof be at least partially formed by the first ramp 310A. The second passage 309 can at a distal portion thereof be at least partially formed by the second ramp 310B.

FIG. 10 shows the distal tip portion 342 with the slot 301, the guide 304, the first ramp 310A, the second ramp 310B, the first passage 307 and the second passage 309. As shown in FIG. 10, the portion of the slot 301 that receives the deformable anchor 150 (FIGS. 8A and 8B) is offset from the first passage 307 but can be generally aligned with the second passage 309. The guide 304, in particular, at least the first ramp 310A can extend distal of the slot 301 where the deformable anchor 150 (FIGS. 8A and 8B) is retained. The first ramp 310A and the second ramp 310B can begin adjacent to or proximal of the slot 301 but can extend in the case of the first ramp 310A at least a distance distal of the portion of the slot 301 that captures the deformable anchor 150 (FIGS. 8A and 8B). The first ramp 310A can be arcuately curved along a longitudinal length (e.g., proximal-distal). Similarly, the second ramp 310B can be arcuately curved along a longitudinal length (e.g., proximal-distal). Such curvatures of the first ramp 310A and the second ramp 310B can be in directions generally opposing one another such that the first ramp 310A and the second ramp 310B (and the first passage 307 and the second passage 309) generally converge toward one another adjacent the distal end of the distal tip portion 342. However, other shapes for the first ramp 310A and/or the second ramp 310B are also contemplated (e.g., curved in three-dimensions, substantially straight, etc.). The first ramp 310A can extend from at or proximal of the slot 301 along the slot 301 and can terminate distal of the portion of the slot 301 that retains the deformable anchor 150 (FIGS. 8A and 8B) adjacent or at the distal tip of the distal tip portion 342.

The curvature or other shape of the first ramp 310A of the guide 304 can deflect (e.g., curve or bend) the drill bit in a desired manner during drilling operation described previously in regard to FIG. 2). Additionally, the guide 304, via at least the second ramp 310B, can be configured to be engaged by the pusher 308 (FIG. 9) during operation when the pusher 308 (FIG. 9) is advanced sufficiently to engage with the guide 304. Thus, as shown in FIGS. 9 and 10, the pusher 308 (FIG. 9) is configured to engage the deformable anchor 150 (FIGS. 8A and 8B) and advance the deformable anchor 150 (FIGS. 8A and 8B) distally from the distal tip portion 342 as directed by the guide 304 which is engaged by the pusher 308 (FIG. 9) when the pusher 308 (FIG. 9) is sufficiently advanced.

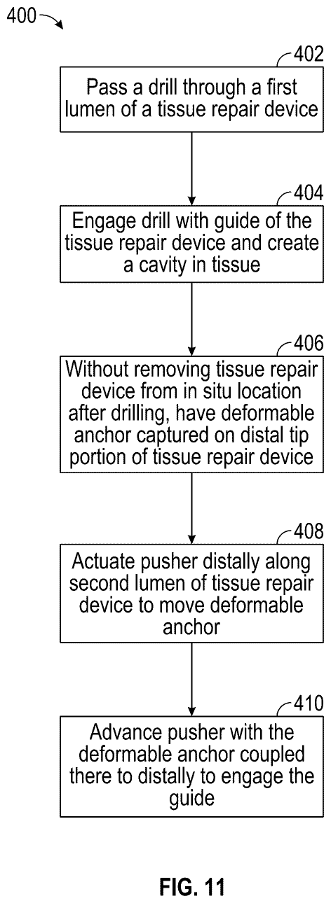

FIG. 11 illustrates method 400 of deploying a deformable anchor from a tissue repair device according to one example. The method 400 can include passing 402 a drill through a first lumen of the tissue repair device. The method 400 can include engaging 404 the drill with a guide of the tissue repair device redirect the drill to cut tissue to create a cavity therein. The method 400 can include, without removing tissue repair device from in situ location after drilling, capturing 406 the deformable anchor on a distal tip portion of the tissue repair device. Thus, the deformable anchor is preloaded onto the tissue repair device even prior to the cutting the tissue to create the cavity. The method 400 can include actuating 408 a pusher distally along a second lumen of the tissue repair device to move the deformable anchor. The method 400 can include advancing 410 the pusher with the deformable anchor coupled thereto distally to engage the guide to direct the deformable anchor into the cavity. Optionally, the method 400 can include that the engaging the drill with the guide causes a bending of the drill along a lateral path that intersects with a path of the pusher during the advancing the pusher. Optionally, the engaging the drill with the guide passes the drill along an arcuate ramp that communicates with at least the first lumen. Optionally, the method 400 can include passing the drill through at least one of a handle or a proximal portion of the pusher of the tissue repair device and into the first lumen.

Many of the FIGURES herein illustrate components of the tissue repair device including the anchors in a highly schematic manner. This is done to better illustrate interaction of various components and the tissue repair device. However, it is recognized that the deformable anchors can have other shapes and can be deformable as discussed above. Similarly, other components can have shapes different from those illustrated herein.

It will be understood that the foregoing are merely examples, and that other deployment schemes and device configurations can also be used. Furthermore, combinations of the various foregoing examples can be used together as desired according to further contemplated examples. It will be understood that one of ordinary skill in the art can modify the foregoing devices to achieve a desirable deployment of the first anchor and the second anchor.

While this invention has been described as having example designs, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

### Claims Related Examples

To further illustrate the devices, systems and related methods disclosed herein, a non-limiting list of examples (referred to as aspects and techniques) is provided below. Each of the following non-limiting examples can stand on its own, or can be combined in any permutation or combination with any one or more of the other examples.

Example 1 is a tissue repair device, optionally comprising: a handle; a shaft assembly coupled to the handle, the shaft assembly having a first lumen and a second lumen, wherein the first lumen is offset from and extends adjacent but separate from the second lumen, and wherein the first lumen is configured to selectively receive a drill; a distal tip portion coupled to or formed by the shaft assembly, wherein the distal tip portion is configured to selectively carry a deformable anchor, and wherein the distal tip portion has a guide; and an actuator moveably coupled to the handle and the shaft assembly, wherein a pusher of the actuator is moveably received within the second lumen, and wherein the pusher is configured to engage the deformable anchor and advance the deformable anchor distally from the distal tip portion as directed by the guide which is engaged by the pusher when the pusher is sufficiently advanced.

In Example 2, the subject matter of Example 1 optionally includes, wherein the shaft assembly includes a first shaft having the first lumen and a second shaft having the second lumen.

In Example 3, the subject matter of Example 2 optionally includes, wherein the distal tip portion comprises an assembly that couples the first shaft with the second shaft.

In Example 4, the subject matter of Example 3 optionally includes, wherein the assembly is enlarged relative to the first shaft and the second shaft.

In Example 5, the subject matter of Examples 1˜4 optionally includes, wherein the guide forms an arcuate ramp that communicates with at least one of the first lumen and the second lumen.

In Example 6, the subject matter of Examples 1-5 optionally includes, wherein the guide is configured to be engaged by and bend the drill when the drill is advanced distally from the distal tip portion.

In Example 7, the subject matter of Examples 1-6 optionally includes, wherein at least one of the handle or the actuator is configured to selectively receive and guide the drill into the first lumen.

Example 8 is a surgical system for repair of tissue, optionally comprising: a drill configured to cut tissue to create a cavity therein; a deformable anchor; and a repair device configured to direct the drill to cut the tissue and direct the deformable anchor into the cavity, the repair device comprising: a handle; a shaft assembly coupled to the handle, the shaft assembly having a first lumen and a second lumen, wherein the first lumen is offset from and extends adjacent but separate from the second lumen, and wherein the first lumen is configured to selectively receive the drill; a distal tip portion coupled to or formed by the shaft assembly, wherein the distal tip portion is configured to selectively carry the deformable anchor, and wherein the distal tip portion has a guide; and an actuator moveably coupled to the handle and the shaft assembly, wherein a pusher of the actuator is moveably received within the second lumen, and wherein the pusher is configured to engage the deformable anchor and advance the deformable anchor distally from the distal tip portion as directed by the guide which is engaged by the pusher when the pusher is sufficiently advanced; wherein the guide is selectively engaged by the drill when the drill is sufficiently advanced, whereby the guide causes a bending of the drill along a lateral path that intersects with a path of the pusher when the pusher is sufficiently advanced to selectively engage the guide.

In Example 9, the subject matter of Example 8 optionally includes, wherein the shaft assembly includes a first shaft having the first lumen and a second shaft having the second lumen.

In Example 10, the subject matter of Example 9 optionally includes, wherein the distal tip portion comprises an assembly that couples the first shaft with the second shaft, and wherein the assembly is enlarged relative to the first shaft and the second shaft.

In Example 11, the subject matter of Examples 8-10 optionally includes, wherein the guide forms an arcuate ramp that communicates with at least one of the first lumen and the second lumen.

In Example 12, the subject matter of Examples 8-11 optionally includes, wherein at least one of the handle or the actuator is configured to selectively receive and guide the drill into the first lumen.

Example 13 is a tissue repair device optionally comprising: a handle; a shaft assembly coupled to the handle, the shaft assembly having a first lumen and a second lumen, wherein the first lumen is offset from and extends adjacent but separate from the second lumen, and wherein the first lumen is configured to selectively receive a drill; a distal tip portion coupled to or formed by the shaft assembly, wherein the distal tip portion is configured to selectively carry a deformable anchor, and wherein the distal tip portion has a guide; and an actuator moveably coupled to the handle and the shaft assembly, wherein a pusher of the actuator is moveably received within the second lumen, and wherein the pusher is configured to engage the deformable anchor and advance the deformable anchor distally from the distal tip portion as directed by the guide; wherein the guide is configured to be selectively engaged by both the drill and the pusher.

In Example 14, the subject matter of Example 13 includes, wherein the guide causes a bending of the drill along a lateral path that intersects with a path of the pusher when the pusher is sufficiently advanced to selectively engage the guide.

In Example 15, the subject matter of Examples 13-14 optionally includes, wherein the shaft assembly includes a first shaft having the first lumen and a second shaft having the second lumen.

In Example 16, the subject matter of Example 15 optionally includes, wherein the distal tip portion comprises an assembly that couples the first shaft with the second shaft, wherein the assembly is enlarged relative to the first shaft and the second shaft.

In Example 17, the subject matter of Examples 13-16 optionally includes, wherein the guide forms an arcuate ramp that communicates with at least one of the first lumen and the second lumen.

In Example 18, the subject matter of Examples 13-17 optionally includes, wherein at least one of the handle or the actuator is configured to selectively receive and guide the drill into the first lumen.

Example 19 is a method of deploying a deformable anchor from a tissue repair device comprising: passing a drill through a first lumen of the tissue repair device; engaging the drill with a guide of the tissue repair device redirect the drill to cut tissue to create a cavity therein; without removing tissue repair device from in situ location after drilling, capturing the deformable anchor on a distal tip portion of the tissue repair device; actuating a pusher distally along a second lumen of the tissue repair device to move the deformable anchor; and advancing the pusher with the deformable anchor coupled thereto distally to engage the guide to direct the deformable anchor into the cavity.

In Example 20, the subject matter of Example 19 optionally includes, wherein engaging the drill with the guide causes a bending of the drill along a lateral path that intersects with a path of the pusher during the advancing the pusher.

In Example 21, the subject matter of Examples 19-20 optionally includes, wherein the engaging the drill with the guide passes the drill along an arcuate ramp that communicates with at least the first lumen.

In Example 22, the subject matter of Examples 19-21 optionally includes, passing the drill through at least one of a handle or a proximal portion of the pusher of the tissue repair device and into the first lumen.

Example 23 is at least one machine-readable medium including instructions that, when executed by processing circuitry, cause the processing circuitry to perform operations to implement of any of Examples 1-22.

Example 24 is an apparatus comprising means to implement of any of Examples 1-22.

Example 25 is a system to implement of any of Examples 1-22.

Example 26 is a method to implement of any of Examples 1-22.

What is claimed is:

1. A tissue repair device, comprising:
a handle;
a shaft assembly coupled to the handle, the shaft assembly having a first lumen and a second lumen, wherein the first lumen is offset from and extends adjacent but separate from the second lumen, and wherein the first lumen is configured to selectively receive a drill;
a distal tip portion coupled to or formed by the shaft assembly, wherein the distal tip portion is configured to selectively carry a deformable anchor, and wherein the distal tip portion has a guide; and
an actuator moveably coupled to the handle and the shaft assembly, wherein a pusher of the actuator is moveably received within the second lumen, and wherein the pusher is configured to engage the deformable anchor and advance the deformable anchor distally from the distal tip portion as directed by the guide which is engaged by the pusher when the pusher is sufficiently advanced.

2. The tissue repair device of claim 1, wherein the shaft assembly includes a first shaft having the first lumen and a second shaft having the second lumen.

3. The tissue repair device of claim 2, wherein the distal tip portion comprises an assembly that couples the first shaft with the second shaft.

4. The tissue repair device of claim 3, wherein the assembly is enlarged relative to the first shaft and the second shaft.

5. The tissue repair device of claim 1, wherein the guide forms an arcuate ramp that communicates with at least one of the first lumen and the second lumen.

6. The tissue repair device of claim 1, wherein the guide is configured to be engaged by and bend the drill when the drill is advanced distally from the distal tip portion.

7. The tissue repair device of claim 1, wherein at least one of the handle or the actuator is configured to selectively receive and guide the drill into the first lumen.

8. A surgical system for repair of tissue, comprising:
a drill configured to cut tissue to create a cavity therein;
a deformable anchor; and
a repair device configured to direct the drill to cut the tissue and direct the deformable anchor into the cavity, the repair device comprising:
a handle;
a shaft assembly coupled to the handle, the shaft assembly having a first lumen and a second lumen, wherein the first lumen is offset from and extends adjacent but separate from the second lumen, and wherein the first lumen is configured to selectively receive the drill;
a distal tip portion coupled to or formed by the shaft assembly, wherein the distal tip portion is configured to selectively carry the deformable anchor, and wherein the distal tip portion has a guide; and
an actuator moveably coupled to the handle and the shaft assembly, wherein a pusher of the actuator is moveably received within the second lumen, and wherein the pusher is configured to engage the deformable anchor and advance the deformable anchor distally from the distal tip portion as directed by the guide which is engaged by the pusher when the pusher is sufficiently advanced;
wherein the guide is selectively engaged by the drill when the drill is sufficiently advanced, whereby the guide causes a bending of the drill along a lateral path that intersects with a path of the pusher when the pusher is sufficiently advanced to selectively engage the guide.

9. The system of claim 8, wherein the shaft assembly includes a first shaft having the first lumen and a second shaft having the second lumen.

10. The system of claim 9, wherein the distal tip portion comprises an assembly that couples the first shaft with the second shaft, and wherein the assembly is enlarged relative to the first shaft and the second shaft.

11. The system of claim 10, wherein the guide forms an arcuate ramp that communicates with at least one of the first lumen and the second lumen.

12. The system of claim 11, wherein at least one of the handle or the actuator is configured to selectively receive and guide the drill into the first lumen.

13. A tissue repair device comprising:
a handle;
a shaft assembly coupled to the handle, the shaft assembly having a first lumen and a second lumen, wherein the first lumen is offset from and extends adjacent but separate from the second lumen, and wherein the first lumen is configured to selectively receive a drill;
a distal tip portion coupled to or formed by the shaft assembly, wherein the distal tip portion is configured to selectively carry a deformable anchor, and wherein the distal tip portion has a guide; and
an actuator moveably coupled to the handle and the shaft assembly, wherein a pusher of the actuator is moveably received within the second lumen, and wherein the pusher is configured to engage the deformable anchor and advance the deformable anchor distally from the distal tip portion as directed by the guide;
wherein the guide is configured to be selectively engaged by both the drill and the pusher.

14. The tissue repair device of claim 13, wherein the guide causes a bending of the drill along a lateral path that intersects with a path of the pusher when the pusher is sufficiently advanced to selectively engage the guide.

15. The tissue repair device of claim 13, wherein the shaft assembly includes a first shaft having the first lumen and a second shaft having the second lumen.

16. The tissue repair device of claim 15, wherein the distal tip portion comprises an assembly that couples the first shaft with the second shaft, wherein the assembly is enlarged relative to the first shaft and the second shaft.

17. The tissue repair device of claim 13, wherein the guide forms an arcuate ramp that communicates with at least one of the first lumen and the second lumen.

18. The tissue repair device of claim 13, wherein at least one of the handle or the actuator is configured to selectively receive and guide the drill into the first lumen.
